(19) United States
(12) Patent Application Publication
(10) Pub. No.: US 2004/0022667 A1
(43) Pub. Date: Feb. 5, 2004

(54) Microbial decontamination and detoxification system and method

(76) Inventors: Sunggyu Lee, Columbia, MO (US); H. Bryan Lanterman, Columbia, MO (US)

Correspondence Address: ALLEN, DYER, DOPPELT, MILBRATH & GILCHRIST, PA, P.O. BOX 3791, ORLANDO, FL, 32802-3791, US

(21) Appl. No.: 10/290,115

(22) Filed: Nov. 7, 2002

Related U.S. Application Data
(60) Provisional application No. 60/344,991, filed on Nov. 7, 2001.

(51) Int. Cl. A61L 9/015

(52) U.S. Cl. 422/4; 422/37; 422/30; 422/123; 422/305; 422/306

(57) ABSTRACT

A system and method for disinfecting and detoxifying an enclosed space includes a generator for producing ClO₂ in aqueous solution via reactions of a hydroxy carboxylic acid, a companion acid that substantially does not react with an alkali metal salt of a chlorite ion, and an alkali metal salt of a chlorite ion. Substantially pure gaseous ClO₂ is stripped through a packed column, the effluent air flow from the stripper containing pure ClO₂. The gaseous ClO₂ product is added to the enclosed space at a predetermined concentration for a predetermined period of time sufficient to substantially destroy airborne microbial contamination therein, after which the enclosed space is evacuated. A second aspect of the present invention is a system and method for disinfecting surfaces, including an apparatus for generating aqueous ClO₂. The aqueous ClO₂ is diluted to a predetermined concentration and applied to a surface desired to be disinfected.

### CROSS-REFERENCE TO RELATED APPLICATION

This application claims priority to provisional application Serial No. 60/344,991, filed Nov. 7, 2001, entitled “Microbial Decontamination and Detoxification System and Method.”

### BACKGROUND OF THE INVENTION

1. Field of the Invention

The present invention relates to system and methods of microbial decontamination and detoxification, and, more particularly, to systems and methods for decontaminating and detoxifying an enclosed airspace and objects therein from airborne and surface-lying microbes.

2. Related Art

At the time of submitting this application, the United States has been facing what is presumed to be a terrorist attack via anthrax deliberately sent through the U.S. postal system. The two most dangerous forms of the disease are the cutaneous and inhalation types. Spore-forming bacteria like Bacillus anthracis can survive without food and water. Anthrax spores can escape through the 10-μm pores of a typical envelope, especially if crushed, such as by a postal sorting machine. Microwave energy does not kill anthrax spores, and the irradiation process that has thus far been proposed requires very expensive machinery.

Chlorine dioxide (ClO₂) is a strong oxidant that has been receiving increased attention as an alternative to chlorine for disinfection purposes. Moreover, chlorine dioxide exhibits a promise of good disinfection performance without the disadvantages of forming large quantities of undesirable chlorinated byproducts since it does not react with hydrocarbons to form chlorinated hydrocarbons.

Chlorine dioxide is known to be an excellent disinfectant as well as a strong oxidizing agent. Its bactericidal, fungicidal, algicidal, bleaching, and deodorizing properties are well documented in the literature. Chlorine dioxide is soluble in water at room temperature (20° C.) to about 2.9 grams ClO₂ per liter of water at 30 mmHg partial pressure of ClO₂, or 8 grams per liter at 80 mmHg partial pressure. ClO₂ is approximately 5 times more soluble in water than chlorine gas (Cl₂). ClO₂ is much more soluble in water than oxygen (O₂), which only has 9.2 mg solubility per liter of water at 20° C. The presence of chlorine dioxide in water is very easily detected by a color change. The color in water changes from yellowish green to orange red as the concentration of ClO₂ increases in water. At low temperatures, chlorine dioxide dissolves in water to a substantially greater extent due to lower vapor pressure, e.g., 12 g/L at 60 mmHg of partial pressure and 10° C. FIG. 1 shows the solubility of ClO₂ in water as a function of temperature. It can be seen that a lower temperature is preferred for higher aqueous solubility.

The boiling point (b.p.) of liquid ClO₂ iS 11° C., and the melting point (m.p.) is −59° C. Gaseous ClO₂ has a density of 2.4 (when taking air as 1.0), and its molecular weight is 67.45 g/mol; i.e., it is a heavier gas than air. If chlorine dioxide is leaked into the air, it will tend to stay low, near the ground, then gradually diffuse into the atmosphere.

Chlorine dioxide (ClO₂) differs from Cl₂ in that ClO₂ does not react with water or ammonia. Also, unlike chlorine, ClO₂ does not produce chlorinated hydrocarbons after reacting with hydrocarbons. In general, ClO₂ is less corrosive to most metallic and nonmetallic substances than chlorine, which is an important advantage. It is also notable that ClO₂ is quite volatile and therefore can be removed easily from aqueous solutions with minimum aeration. Concentrations of ClO₂ in air above 11% can be mildly explosive. Due to the chlorine dioxide's relative instability and volatility, storage and transportation seem intuitively less economical, even though it is conceivable to store it in a compressed container. In this regard, the strategy of ClO₂ production can be twofold, viz., either on-site production of pure aqueous ClO₂ or high-purity compressed ClO₂.

A method and apparatus for the production of chlorine dioxide, and for treating water and/or wastewater using aqueous chlorine dioxide, has been disclosed in co-owned U.S. Pat. Nos. 5,855,861 and 6,051,135, the disclosures of which are incorporated herein by reference. These inventions are directed to an economic and efficient apparatus for producing chlorine dioxide that does not also produce hazardous by-products (e.g., chlorine or chlorous acid), as well as substantial amounts of unusable salts (e.g., sodium chloride, sodium lactate), and to a method of producing chlorine dioxide that does not suffer from a slow rate of reaction and low concentrations of chlorine dioxide.

As disclosed in the above-referenced inventions, an apparatus for making chlorine dioxide and for disinfecting water or wastewater includes mechanisms for feeding solutions containing a hydroxy carboxylic acid, a companion acid, and an alkali metal of a chlorite ion to a reaction vessel, a reaction vessel, and a mechanism for withdrawing product solution from the reaction vessel. The apparatus also can include a stripping unit whereby product solution is contacted with an inert gas to produce a product gas, and an absorbing unit whereby the product gas is contacted with an aqueous medium to produce an aqueous solution of chlorine dioxide.

The apparatus disclosed in the '861 and '135 patents has received National Sanitation Foundation International 60 and 61 certification, and has been shown to produce chlorine dioxide that is pure and free from any contamination of by-products and leachates.

### SUMMARY OF THE INVENTION

The system and method of the present invention disinfects and detoxifies an enclosed space from airborne microbes, and also disinfects and detoxifies surfaces. The invention further provides a system and method that generate and utilize aqueous and/or gaseous chlorine dioxide with the use of a chlorine dioxide generator situated on site, the generator preferably being portable and inexpensive.

An exemplary beneficial aspect of the system and method is that free chlorine is not involved, either as a raw material or as a by-product; another is that other chlorine-containing compounds in the product chlorine dioxide are not produced.

These and other aspects are achieved by a first embodiment of the present invention, a system and method for disinfecting and detoxifying an enclosed space. The system comprises a ClO₂ generator substantially as disclosed in the embodiments of the above-referenced '861 and '135 patents, wherein the ClO₂ is generated in aqueous solution via reactions of a hydroxy carboxylic acid, a companion acid that substantially does not react with an alkali metal salt of a chlorite ion, and an alkali metal salt of a chlorite ion. Substantially pure gaseous ClO₂ is stripped by controlled air flow through a packed column, the effluent air flow from the stripper containing pure ClO₂.

The gaseous ClO₂ product is added to the enclosed space at a predetermined concentration for a predetermined period of time sufficient to substantially destroy airborne microbial contamination therein.

After the predetermined period of time, the air in the enclosed space is evacuated through a scrubber system comprising a packed column for transferring gaseous ClO₂ to water in the scrubber to form aqueous ClO₂ solution and release substantially ClO₂-free and disinfected air for return to the enclosed space as desired or to the atmosphere.

A second aspect of the present invention is a system and method for disinfecting surfaces. The system for this application comprises an apparatus substantially identical with that disclosed in the '861 and '135 patents for generating aqueous ClO₂. The aqueous ClO₂ is diluted to a predetermined concentration and applied to a surface desired to be disinfected.

Pure chlorine dioxide is believed to be more potent in bacterial treatment than any other chlorine compound. The systems and methods of the present invention involve tested and approved components, and are not believed to cause any safety or health concerns regarding fire, explosion, strong acid involvement, or undesirable by-products.

Further objects, features and advantages of the present invention will become apparent from the detailed description of the preferred embodiments that follows.

### BRIEF DESCRIPTION OF THE DRAWING

FIG. 1 (prior art) is a plot of the solubility of ClO₂ in water as a function of temperature.

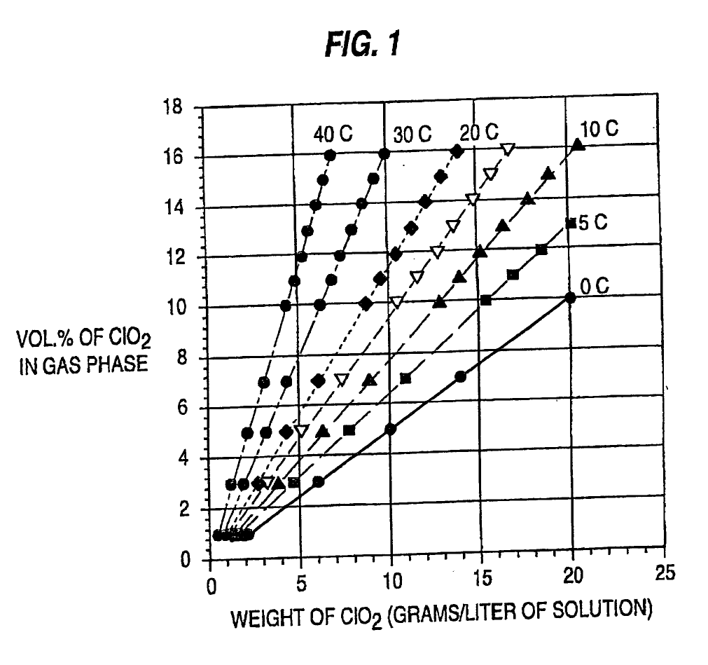

FIG. 2 is a schematic illustration of a system for disinfecting and detoxifying an enclosed space.

FIG. 3 is a schematic illustration of a system for degassing the enclosed space.

FIG. 4 is a schematic illustration of a system for producing chlorine dioxide.

### DETAILED DESCRIPTION OF THE PREFERRED EMBODIMENTS

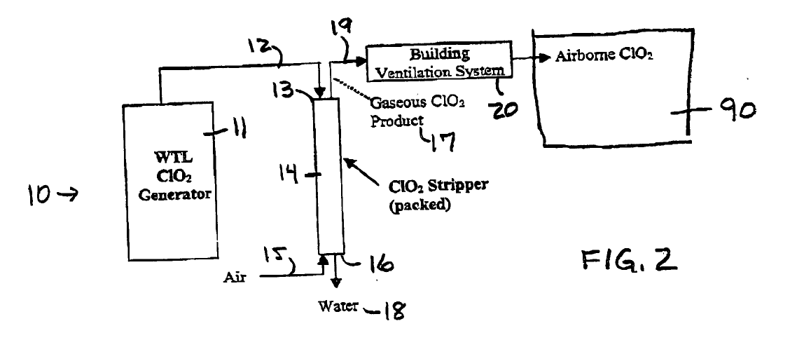

A detailed description of the preferred embodiments of the present invention will now be presented with reference to FIGS. 2-4.

System and Method for Disinfecting and Decontaminating an Enclosed Space

In a first embodiment of the present invention, a system 10 (FIG. 2) is provided for disinfecting and decontaminating an enclosed space 90 containing an airborne contaminant, such as, but not intended to be limited to, airborne anthrax spores. The system 10 comprises a ClO₂ generator 11 such as disclosed in the aforementioned '861 and '135 patents for producing aqueous ClO₂ from sodium chlorite, lactic acid, and acetic acid. A line 12 directs the aqueous ClO₂ to a first end 13 of a packed ClO₂ stripper 14, wherein controlled airflow 15 entering from a second end 16 of the stripper 14 takes gaseous ClO₂ 17 into the flow, with stripped water 18 exiting from the stripper's second end 16. In a preferred embodiment, the effluent air flow 19 has approximately 6% by volume pure ClO₂. The stripper 14 of the present system 10 may comprise, for example, a device such as described in the '861 and '135 patents.

If the enclosed space 90 comprises, for example, a building, the effluent air flow 19 may be directed to the building's ventilation system 20, thereby providing a controlled dosage of ClO₂ in every part of the building 90 desired to be decontaminated using devices known in the art for controlling air flow. In an exemplary embodiment, a typical concentration needed for substantially total destruction of anthrax is approximately 10-500 ppm, preferably 50-100 ppm, with a dwell time of approximately 10-200 min., preferably 30-60 min. The dwell time should be long enough to destroy bacteria while short enough to prevent ClO₂ from going through gaseous decomposition into Cl₂ and O₂.

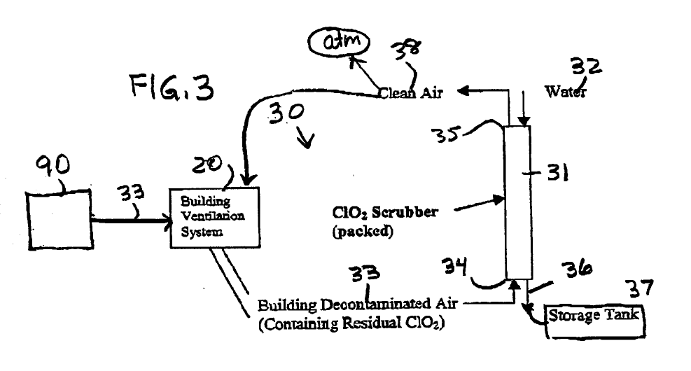

After the predetermined dwell time for ClO₂ treatment, it is preferred to evacuate the ClO₂-laden air therewithin by, for example, a reverse flow through the building ventilation system 20, as shown in FIG. 3. Removal of gaseous ClO₂ from building air is accomplished in an exemplary embodiment by means of a system 30 comprising a component of the apparatus disclosed in the '861 and '135 patents, a packed ClO₂ scrubber 31, which uses water 32 to absorb ClO₂ from the airstream 33 exiting from the building's ventilation system 20.

In this system 30, the decontaminated air 33 enters a first end 34 of the scrubber 31 and passes therethrough to a second end 35. Counter-flowing water 32 enters at the second end 35, exits at the first end 34 as aqueous ClO₂ 36, which is piped to a storage tank 37. Depending upon the final application, the scrubber water may be mixed with an alkaline solution such as sodium hydroxide solution in order to neutralize the prescrubbed air. The scrubbed air 38 may be vented to the atmosphere or recycled into the building's ventilation system 20.

Air stripped through the system 10, as an exemplary embodiment, can reach 300 liters of air/h at 150 mg pure ClO₂ per liter of air, or at 6 vol % concentration.

If the air concentration of ClO₂ is desired to be 100 ppm by volume, as an exemplary calculation, the system 10 is capable of generating ClO₂-containing air at 0.18×10⁶ liters of air/h, or 180,000 liters/h.

System and Method for Disinfecting a Surface

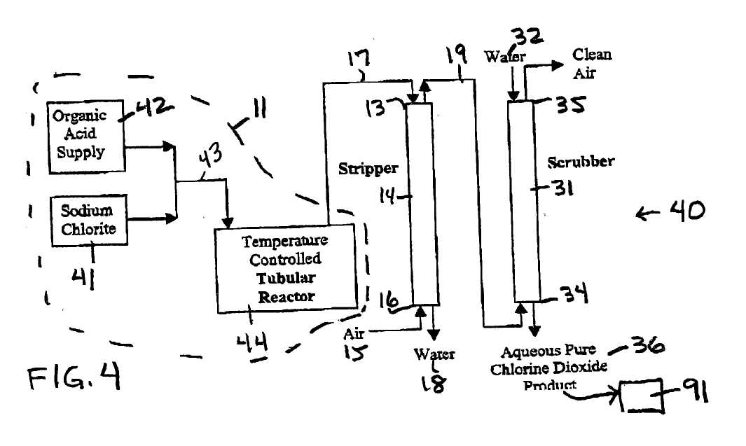

In a second embodiment of the present invention, a system 40 (FIG. 4) is provided for disinfecting a surface 91 containing a contaminant, such as, but not intended to be limited to, anthrax spores. The system 40 comprises a ClO₂ generator 11 such as disclosed in the aforementioned '861 and '135 patents for producing aqueous ClO₂ from sodium chlorite 41 and an organic acid 42, such as lactic acid and acetic acid. A line 43 directs the mixture to a temperature-controlled tubular reactor 44, which produces aqueous ClO₂. The aqueous ClO₂ enters the first end 13 of a packed ClO₂ stripper 14, wherein controlled airflow 15 entering from a second end 16 of the stripper 14 takes gaseous ClO₂ 17 into the flow, with stripped water 18 exiting from the stripper's second end 16.

The effluent air flow 19 enters the first end 34 of the scrubber 31 and passes therethrough to a second end 35. Counter-flowing water 32 enters at the second end 35, exits at the first end 34 as aqueous ClO₂ 36.

This system 40 is known to produce pure aqueous ClO₂ in concentrations up to 8000 ppm, substantially free from by-products and other chlorine-containing compounds. It is believed that the disinfection capability of this solution is essentially limitless, without having the potential for causing harmful side effects on humans or other animals.

To achieve disinfection according to this aspect of the invention, the product ClO₂ is typically diluted commensurate with the intended application. In the exemplary use for a spray-and-scrub application for anthrax detoxification, a concentration of pure ClO₂ in water of ˜50 ppm is believed optimal, which is approximately equivalent to 50 mg/liter. This level is, of course, not intended to be limiting on the present invention. This concentration has sufficient potency to substantially destroy anthrax spores in less than 1 min.

Spray solutions may be applied, for example, to a wet cloth, or by other means, for decontaminating a targeted area and object such as a desktop, an area under furniture, a shelf, office equipment, and any other surface, inner or outer, desired to be treated. A typical concentration for such application of pure ClO₂ aqueous solution ranges from 10-200 ppm, preferably 50-100 ppm.

The system 40 in an exemplary embodiment has a capacity of 1.5 gal solution/h (6 liters/h), 7000-8000 mg pure ClO₂/liter of water. The hourly ClO₂ production of this system 40 is 6 liters aqueous solution/h at 7500 mg/liter aqueous solution=45,000 mg pure ClO₂/h.

If dilute solutions of aqueous ClO₂ are desired, the system 40 is capable of producing 900 liters (˜230 gal) of 50-ppm ClO₂ solution per hour.

In the foregoing description, certain terms have been used for brevity, clarity, and understanding, but no unnecessary limitations are to be implied therefrom beyond the requirements of the prior art, because such words are used for description purposes herein and are intended to be broadly construed. Moreover, the embodiments of the system and method illustrated and described herein are by way of example, and the scope of the invention is not limited to the exact details of construction.

Having now described the invention, the construction, the operation and use of preferred embodiments thereof, and the advantageous new and useful results obtained thereby, the new and useful constructions, and reasonable mechanical equivalents thereof obvious to those skilled in the art, are set forth in the appended claims.

What is claimed is:

1. A system for substantially disinfecting and detoxifying an enclosed space comprising:
a generator for producing a chlorine dioxide solution from a hydroxy carboxylic acid, a companion acid that substantially does not react with an alkali metal salt of a chlorite ion, and an alkali metal salt of a chlorite ion;
a stripper for stripping chlorine dioxide gas from the produced chlorine dioxide solution; and
duct means for directing the stripped chlorine dioxide gas into the enclosed space at a predetermined concentration and for a predetermined time sufficient to substantially destroy airborne microbial contamination therein.

2. The system recited in claim 1, wherein the hydroxy carboxylic acid is capable of temporarily transferring chlorine from an alkali metal salt of chlorite ion, but substantially does not form a salt with an alkali metal salt of a chlorite ion.

3. The system recited in claim 1, wherein the generator comprises a vessel for mixing an aqueous solution of the hydroxy carboxylic acid and the companion acid with the alkali metal salt to yield an aqueous reaction solution.

4. The system recited in claim 3, wherein the generator is adapted to maintain a pH of the aqueous reaction solution at a value of ≦4 while the mixing is occurring in the vessel.

5. The system recited in claim 1, wherein the hydroxycarboxylic acid is lactic acid, the companion acid is acetic acid, and the alkali metal salt is sodium chlorite.

6. The system recited in claim 1, wherein the stripper comprises a packed column.

7. The system recited in claim 1, wherein the chlorine dioxide gas exiting the stripper contains approximately 6% chlorine dioxide by volume.

8. The system recited in claim 1, further comprising means for controlling the predetermined concentration to a level between 10 and 500 ppm.

9. The system recited in claim 1, further comprising means for controlling the predetermined concentration to a level between 50 and 100 ppm.

10. The system recited in claim 1, further comprising means for evacuating the enclosed space to remove chlorine dioxide gas therefrom.

11. The system recited in claim 10, wherein the evacuating means comprises a scrubber system.

12. The system recited in claim 11, wherein the scrubber system comprises a packed column for transferring gaseous chlorine dioxide to water to form an aqueous chlorine dioxide solution.

13. The system recited in claim 12, further comprising duct means for directing air substantially free of chlorine dioxide to at least one of the enclosed space and the atmosphere.

14. The system recited in claim 12, further comprising a storage tank for receiving the aqueous chlorine dioxide solution from the evacuating means packed column.

15. The system recited in claim 10, wherein the evacuating means comprises means for imposing a dwell time of approximately 10-200 min.

16. The system recited in claim 10, wherein the evacuating means comprises means for imposing a dwell time of approximately 30-60 min.

17. A method for substantially disinfecting and detoxifying an enclosed space comprising the steps of:
producing a chlorine dioxide solution from a hydroxy carboxylic acid, a companion acid that substantially does not react with an alkali metal salt of a chlorite ion, and an alkali metal salt of a chlorite ion;
stripping chlorine dioxide gas from the produced chlorine dioxide solution; and
directing the stripped chlorine dioxide gas into the enclosed space at a predetermined concentration and for a predetermined time sufficient to substantially destroy airborne microbial contamination therein.

18. The method recited in claim 17, wherein the hydroxy carboxylic acid is capable of temporarily transferring chlorine from an alkali metal salt of chlorite ion, but substantially does not form a salt with an alkali metal salt of a chlorite ion.

19. The method recited in claim 17, wherein the producing step comprises mixing an aqueous solution of the hydroxy carboxylic acid and the companion acid with the alkali metal salt to yield an aqueous reaction solution.

20. The method recited in claim 19, further comprising the step of maintaining a pH of the aqueous reaction solution at a value of ≦4 during the mixing step.

21. The method recited in claim 17, wherein the hydroxycarboxylic acid is lactic acid, the companion acid is acetic acid, and the alkali metal salt is sodium chlorite.

22. The method recited in claim 17, wherein the stripping step comprises passing the produced chlorine dioxide solution through a packed column.

23. The method recited in claim 22, wherein the chlorine dioxide gas exiting the column contains approximately 6% chlorine dioxide by volume.

24. The method recited in claim 17, further comprising the step of controlling the predetermined concentration to a level between 10 and 500 ppm.

25. The method recited in claim 17, further comprising the step of controlling the predetermined concentration to a level between 50 and 100 ppm.

26. The method recited in claim 17, further comprising the step of evacuating the enclosed space to remove chlorine dioxide gas therefrom following the directing step.

27. The method recited in claim 26, wherein the evacuating step comprises passing the evacuated air from the enclosed space through a scrubber.

28. The method recited in claim 27, wherein the scrubber comprises a packed column for transferring gaseous chlorine dioxide to water to form an aqueous chlorine dioxide solution.

29. The method recited in claim 28, further comprising the step of directing air substantially free of chlorine dioxide exiting the packed column to at least one of the enclosed space and the atmosphere.

30. The method recited in claim 28, further comprising the step of storing aqueous chlorine dioxide solution exiting the evacuating means packed column.

31. The method recited in claim 26, further comprising the step of imposing a dwell time for chlorine dioxide gas in the enclosed space of approximately 10-200 min.

32. The method recited in claim 26, further comprising the step of imposing a dwell time for chlorine dioxide gas in the enclosed space of approximately 30-60 min.

33. A system for disinfecting a surface comprising:
a generator for producing a chlorine dioxide solution from a hydroxy carboxylic acid, a companion acid that substantially does not react with an alkali metal salt of a chlorite ion, and an alkali metal salt of a chlorite ion;
a stripper for stripping chlorine dioxide gas from the produced chlorine dioxide solution;
a scrubber for producing aqueous chlorine dioxide from the chlorine dioxide gas exiting the stripper;
means for applying the aqueous chlorine dioxide solution exiting the scrubber to a surface to substantially destroy microbial contamination thereon.

34. The system recited in claim 33, wherein the hydroxy carboxylic acid is capable of temporarily transferring chlorine from an alkali metal salt of chlorite ion, but substantially does not form a salt with an alkali metal salt of a chlorite ion.

35. The system recited in claim 33, wherein the generator comprises a vessel for mixing an aqueous solution of the hydroxy carboxylic acid and the companion acid with the alkali metal salt to yield an aqueous reaction solution.

36. The system recited in claim 35, wherein the generator is adapted to maintain a pH of the aqueous reaction solution at a value of ≦4 while the mixing is occurring in the vessel.

37. The system recited in claim 33, wherein the hydroxycarboxylic acid is lactic acid, the companion acid is acetic acid, and the alkali metal salt is sodium chlorite.

38. The system recited in claim 33, wherein the stripper comprises a packed column.

39. The system recited in claim 33, wherein the aqueous chlorine dioxide solution exiting the scrubber has a chlorine dioxide concentration up to 8000 ppm.

40. The system recited in claim 33, further comprising means for diluting the aqueous chlorine dioxide solution exiting the scrubber to a level of 10-200 ppm.

41. A method for disinfecting a surface comprising the steps of:
producing a chlorine dioxide solution from a hydroxy carboxylic acid, a companion acid that substantially does not react with an alkali metal salt of a chlorite ion, and an alkali metal salt of a chlorite ion;
stripping chlorine dioxide gas from the produced chlorine dioxide solution;
forming aqueous chlorine dioxide from the stripped chlorine dioxide gas;
applying the formed aqueous chlorine dioxide solution to a surface to substantially destroy microbial contamination thereon.

42. The method recited in claim 41, wherein the hydroxy carboxylic acid is capable of temporarily transferring chlorine from an alkali metal salt of chlorite ion, but substantially does not form a salt with an alkali metal salt of a chlorite ion.

43. The method recited in claim 41, wherein the producing step comprises mixing an aqueous solution of the hydroxy carboxylic acid and the companion acid with the alkali metal salt to yield an aqueous reaction solution.

44. The method recited in claim 43, further comprising the step of maintaining a pH of the aqueous reaction solution at a value of ≦4 during the mixing step.

45. The method recited in claim 41, wherein the hydroxycarboxylic acid is lactic acid, the companion acid is acetic acid, and the alkali metal salt is sodium chlorite.

46. The method recited in claim 41, wherein the stripping step comprises directing the produced chlorine dioxide solution through a packed column.

47. The method recited in claim 41, wherein the formed aqueous chlorine dioxide solution has a chlorine dioxide concentration up to 8000 ppm.

48. The method recited in claim 41, further comprising the step of diluting the aqueous chlorine dioxide solution exiting the scrubber to a level of 10-200 ppm.
